(19) United States
(12) Patent Application Publication
(10) Pub. No.: US 2013/0059019 A1
(43) Pub. Date: Mar. 7, 2013

(54) METHODS AND COMPOSITIONS FOR TREATING INFLAMMATION OF SKIN

(71) Applicants: Harry J. Leighton, Rockport, ME (US); Crist J. Frangakis, Chapel Hill, NC (US)

(72) Inventors: Harry J. Leighton, Rockport, ME (US); Crist J. Frangakis, Chapel Hill, NC (US)

(73) Assignee: Exodus Life Sciences Limited Partnership, Chapel Hill, NC (US)

(21) Appl. No.: 13/517,008

(22) PCT Filed: Dec. 17, 2010
(86) PCT No.: PCT/US10/61051

§ 371 Date: Nov. 27, 2012

Related U.S. Application Data
(60) Provisional application No. 61/287,980, filed on Dec. 18, 2009.

(51) Int. Cl. A61K 31/335; A61P 29/00; A61P 17/04; A61P 17/06; A61P 25/24; A61K 36/9068; A61P 31/10; A61P 31/04; A61P 25/04; A61P 17/00; A61P 17/02

(52) U.S. Cl. 424/736; 514/450

(57) ABSTRACT

The invention features methods of treating a subject suffering from a herpes simplex virus-induced inflammation by topically applying a composition including an effective amount of an antihistamine. The invention also features methods of treating inflammation by topically applying a base composition including essential extracts, either with or without one or more therapeutic agents. Also provided are compositions formulated for topical administration including a base composition, as well as kits including the composition.

### CROSS REFERENCE TO RELATED APPLICATIONS

This application claims the benefit of U.S. Provisional Application No. 61/287,980, filed Dec. 18, 2009.

### BACKGROUND OF THE INVENTION

This invention relates to methods and compositions for treating inflammation of skin. In particular, methods are provided that involve topical application of a base composition to treat inflammation, such as that resulting from a viral and/or bacterial infection. As described herein, the base composition can be used alone or in combination with one or more therapeutic agents.

Skin trauma can be caused by a variety of factors, including viral infection, bacterial infection, exposure to heat, chemical irritants, and excessive sun exposure. These factors cause painful skin conditions associated with edema, blistering, itching, and swelling of local tissues.

For example, an active replicating, Herpes Simplex Virus 1 (HSV-1) will induce the phenotype commonly referred to as a cold sore. A cold sore is an area of erythema, redness, blistering, and itching. Viral replication causes cellular damage that induces the immune system to react. Histamine is released from local mast cells and induces swelling and redness and signals in elements of the circulating immune system. This multi-component immune response prolongs the duration of the cold sore outbreak (usually 7-10 days). Viral replication is the initiating factor; the immune response protracts the cold sore phenotype outbreak. In a similar manner, genital lesions and associated pain, itching, and edema are the result of activation of Herpes Simplex Virus-2 (HSV-2).

Treatment of these lesions is usually by the oral ingestion or topical application of specific, potent antiviral agents, including but not limited to acyclovir, valcyclovir, peniclovir, foscarnet, and docosanol. Surprisingly, while these treatments are viral specific and non-toxic, efficacy is limited and can be slow despite substantial skin penetration. A conventional topical antiviral medication can take well over 6 days of multiple applications to reverse the cosmetic appearance induced by the initial viral replication of the virus in the lip or genital area. Effectiveness by the oral route of administration is largely a timing issue. If one catches the virus in the prodromal stage, then a virus outbreak may be prevented or the symptoms may be lessened. In many cases, the subject misses this window of opportunity. Once the virus starts replicating, tissue injury and immune response cannot be avoided.

### SUMMARY OF THE INVENTION

Due to the multi-component nature of a cold sore or a genital lesion, the present invention employs a multi-component treatment that includes a base composition, which reduces swelling and itching and enhances healing rate, that can be used alone or in combination with certain mechanism-based therapeutic agents in a topical formulation. In one particular embodiment, the base composition includes: beeswax, castor seed oil, hydrogenated castor oil, carnauba wax, sweet almond oil, caprylic/capric triglycerides, lanolin, tocopherol acetate, hempseed oil, an herbal infused oil, and/or the following essential extracts: rosemary (Rosmarinus officinalis), basil (Ocimum basilicum), ginger (Zingiber officinale Roscoe), sweet orange (Citrus sinensis), Geranium Egypt (Pelargonium graveolens), lemon (Citrus limonum), peppermint (Mentha piperita), Tea Tree (Melaleuca alternifolia), vanilla infused oil, and/or stevia (Eupatorium rebaudianum).

In a first aspect, the invention features a method of treating a subject suffering from a herpes simplex virus-induced inflammation, the method including topically applying to an affected area of the subject a composition including an effective amount of an antihistamine. In one embodiment, the inflammation is a Herpes Simplex Virus-1 (HSV-1)-induced inflammation. In another embodiment, the inflammation is a Herpes Simplex Virus-2 (HSV-2)-induced inflammation.

In one embodiment of the first aspect, the method includes topically applying to an affected area of the subject a composition including an antihistamine selected from the group consisting of doxepin, amitriptyline, triprolidine, acrivastine, and diphenhydramine. In a further embodiment, the composition further includes an ion channel blocking agent and an antiviral agent.

In a second aspect, the invention features a method of treating inflammation of skin in a subject, the method including topically administering to an affected area of the subject a base composition in an amount that is effective to treat the inflammation, where the base composition includes 70% to 95% (w/w) of one or more waxes, 5% to 10% (w/w) of one or more essential extracts, 0.1% to 1.0% (w/w) of a thickener, and 0.1% to 0.5% (w/w) of an antioxidant.

In one embodiment of the second aspect, the inflammation is associated with one or more of pruritus, viral-induced inflammation, eczema, shingles, psoriasis, atopic dermatitis, bacterial-induced inflammation, fungal-induced inflammation, burns, laceration damage, and acute injuries. In another embodiment, the inflammation is viral-induced inflammation (e.g., the viral-induced inflammation is associated with a cold sore).

In an embodiment of the second aspect, the method includes topically administering a base composition including one or more waxes selected from the group consisting of beeswax, carnauba wax, and lanolin. In another embodiment, the base composition includes one or more essential extracts selected from the group consisting of rosemary oil (Rosmarinus officinalis), basil oil (Ocimum basilicum), ginger oil (Zingiber officinale Roscoe), sweet orange oil (Citrus sinensis), Geranium Egypt oil (Pelargonium graveolens), lemon oil (Citrus limonum), peppermint oil (Mentha piperita), Tea Tree oil (Melaleuca alternifolia), vanilla infused oil, stevia (Eupatorium rebaudianum), sweet almond oil, castor seed oil, hydrogenated castor oil, and hempseed oil. In yet another embodiment, the base composition includes a thickener, where the thickener is a caprylic/capric triglyceride. In another embodiment, the base composition includes an antioxidant, where the antioxidant is tocoperol or a derivative thereof. In a further embodiment, the base composition further includes 5% to 10% (w/w) of an herbal infused oil (e.g., coconut oil infused with lemon balm (Melissa officinalis), calendula flowers (Calendula officinalis), green tea gunpowder (Camellia sinensis), and green rooibos (Aspalatus linearis)).

In one embodiment of the second aspect, the base composition further includes one or more therapeutic agents selected from the group consisting of an antibacterial agent (e.g., demeclocycline, chlortetracycline, oxytetracycline, tetracycline, chloramphenicol, neomycin, gentamicin, amikacin, clindamycin, nadifloxacin, streptogramin, virginiamycin, rifamycin, rifaximin, fusidic acid, bacitracin, tyrothricin, and mupirocin), an antifungal agent (e.g., terbinafine hydrochloride, clotrimazole, ketoconazole, nystatin, natamycin, hachimycin, pecilocin, mepartricin, pyrrolnitrin, griseofulvin, miconazole, econazole, clomidazole, isoconazole, tiabendazole, tioconazole, sulconazole, bifonazole, oxiconazole, fenticonazole, omoconazole, sertaconazole, fluconazole, flutrimazole, enilconazole, bromochlorosalicylanilide, methylrosaniline, tribromometacresol, undecylenic acid, polynoxylin, 2-(4-chlorphenoxy)-ethanol, chlorphenesin, ticlatone, sulbentine, ethyl hydroxybenzoate, haloprogin, salicylic acid, selenium sulfide, ciclopirox, amorolfine, dimazole, tolnaftate, tolciclate, flucytosine, naftifine, butenafine, undecylenic acid, bronopol, and bensuldazic acid), an antihistamine (e.g., a tricyclic antidepressant, such as doxepin or amitriptyline or a pharmaceutically acceptable salt thereof; an ethanolamine agent, such as diphenhydramine; an ethylenediamine agent; an alkylamine agent, such as a triprolidine, acrivastine, or chlorpheniramine; a piperazine agent; a phenothiazine agent, such as promethazine or chlorpromazine; and a piperidine agent, such as cyproheptadine), an antiinflammatory agent (e.g., aspirin, diclofenac, ibuprofen, ketoprofen, and naproxen), an antiviral agent (e.g., acyclovir, cidofovir, docosanol, famciclovir, foscarnet, fomivirsen, ganciclovir, idoxuridine, penciclovir, peramivir, trifluridine, valacyclovir, vidarabine, lamivudine, and ribavirin), an ion channel blocking agent (e.g., a sodium channel blocking agent, such as benzocaine, bupivacaine, lidocaine, etidocaine, mepivacaine, pramoxine, prilocaine, procaine, proparacaine, ropivacaine, tetracaine; or an acid sensitive ion channel blocking agent, such as amiloride or derivatives or pharmaceutically acceptable salts thereof), and an opioid (e.g., morphine, codeine, meperidine, and oxycodone).

In another embodiment of the second aspect, the method includes one or more therapeutic agents, where one or more antihistamines selected from the group consisting of a tricyclic antidepressant, an ethanolamine agent, an ethylenediamine agent, an alkylamine agent, a piperazine agent, a phenothiazine agent, and a piperidine agent (e.g., the tricyclic antidepressant and the ethanolamine agent, such as doxepin or a pharmaceutically acceptable salt thereof and diphenhydramine; or the tricyclic antidepressant and the alkylamine agent, such as doxepin or a pharmaceutically acceptable salt thereof and the alkylamine agent is triprolidine or acrivastine). In a particular embodiment, the method includes one or more antihistamines selected from the group consisting of a tricyclic antidepressant, an ethanolamine agent, an ethylenediamine agent, an alkylamine agent, a piperazine agent, a phenothiazine agent, and a piperidine agent; and one or more antiinflammatory agents (e.g., the antihistamine is doxepin or a pharmaceutically acceptable salt thereof and the antiinflammatory agent is ketoprofen). In another particular embodiment, the method includes one or more antihistamines selected from the group consisting of a tricyclic antidepressant, an ethanolamine agent, an ethylenediamine agent, an alkylamine agent, a piperazine agent, a phenothiazine agent, and a piperidine agent; and one or more antiviral agents (e.g., the antihistamine is doxepin or a pharmaceutically acceptable salt thereof and the one or more antiviral agents are selected from the group consisting of acyclovir and valacyclovir). In yet another particular embodiment, the method includes one or more antihistamines selected from the group consisting of a tricyclic antidepressant, an ethanolamine agent, an ethylenediamine agent, an alkylamine agent, a piperazine agent, a phenothiazine agent, and a piperidine agent; and one or more ion channel blocking agents selected from the group consisting of a sodium channel blocking agent and an acid sensitive ion channel blocking agent (e.g., the antihistamine is doxepin or a pharmaceutically acceptable salt thereof and the one or more ion channel blocking agents are selected from the group consisting of lidocaine, benzocaine, and tetracaine). In a further particular embodiment, the method includes one or more antihistamines selected from the group consisting of a tricyclic antidepressant, an ethanolamine agent, an ethylenediamine agent, an alkylamine agent, a piperazine agent, a phenothiazine agent, and a piperidine agent; one or more ion channel blocking agents selected from the group consisting of a sodium channel blocking agent and an acid sensitive ion channel blocking agent; and one or more antiviral agents selected from the group consisting of acyclovir, cidofovir, docosanol, famciclovir, foscarnet, fomivirsen, ganciclovir, idoxuridine, penciclovir, peramivir, trifluridine, valacyclovir, vidarabine, lamivudine, and ribavirin.

In one embodiment of the second aspect, the base composition includes 0.1% to 30% (w/w) of one or more therapeutic agents (e.g., 1% to 10% (w/w) of one therapeutic agent, or 10% to 25% (w/w) of two or more therapeutic agents). In a particular embodiment, the base composition includes 1% to 10% (w/w) of doxepin or a pharmaceutically acceptable salt thereof In another particular embodiment, the base composition includes 1% to 10% (w/w) of doxepin or a pharmaceutically acceptable salt thereof and 1% to 10% (w/w) of acyclovir or valacyclovir.

In a third aspect, the invention features a composition formulated for topical administration including a base composition, where the base composition includes 70% to 95% (w/w) of one or more waxes, 5% to 10% (w/w) of one or more essential extracts, 0.1% to 1.0% (w/w) of a thickener, and 0.1% to 0.5% (w/w) of an antioxidant.

In one embodiment of the third aspect, the base composition includes 70% to 95% (w/w) of beeswax, carnauba wax, and lanolin, 5% to 10% (w/w) of one or more essential extracts, 0.1% to 1.0% (w/w) of caprylic/capric triglycerides, and 0.1% to 0.5% (w/w) of tocopherol acetate. In another embodiment, the one or more essential extracts are selected from the group consisting of rosemary oil (Rosmarinus officinalis), basil oil (Ocimum basilicum), ginger oil (Zingiber officinale Roscoe), sweet orange oil (Citrus sinensis), Geranium Egypt oil (Pelargonium graveolens), lemon oil (Citrus limonum), peppermint oil (Mentha piperita), Tea Tree oil (Melaleuca alternifolia), vanilla infused oil, stevia (Eupatorium rebaudianum), sweet almond oil, castor seed oil, hydrogenated castor oil, and hempseed oil.

In a further embodiment of the third aspect, the base composition further includes 5% to 10% (w/w) of an herbal infused oil (e.g., coconut oil infused with lemon balm (Melissa officinalis), calendula flowers (Calendula officinalis), green tea gunpowder (Camellia sinensis), and green rooibos (Aspalatus linearis)).

In another embodiment of the third aspect, the composition further includes one or more therapeutic agents selected from the group consisting of an antibacterial agent, an antifungal agent, an antihistamine, an antiinflammatory agent, an antiviral agent, an ion channel blocking agent, and an opioid.

In one embodiment of the third aspect, the composition includes 0.1% to 30% (w/w) of one or more therapeutic agents (e.g., 1% to 10% (w/w) of one therapeutic agent or 10% to 25% (w/w) of two or more therapeutic agents). In a particular embodiment, the composition includes one or more antihistamines (e.g., 1% to 25% (w/w) of one or more of doxepin, amitriptyline, triprolidine, acrivastine, or diphenhydramine or a pharmaceutically acceptable salt thereof). In another particular embodiment, the composition includes the antihistamine and the antiinflammatory agent (e.g., the antihistamine is 1% to 10% (w/w) of doxepin or a pharmaceutically acceptable salt thereof and the antiinflammatory agent is 1% to 10% (w/w) of ketoprofen). In yet another particular embodiment, the composition includes the antihistamine and the antiviral agent (e.g., the antihistamine is from 1% to 10% (w/w) doxepin or a pharmaceutically acceptable salt thereof and the antiviral agent is from 5% to 15% (w/w) acyclovir or valacyclovir). In a further particular embodiment, the composition includes the antihistamine and the ion channel blocking agent (e.g., the antihistamine is from 1% to 10% (w/w) doxepin or a pharmaceutically acceptable salt thereof and the ion channel blocking agent is from 5% to 15% (w/w) lidocaine, benzocaine, bupivacaine, etidocaine, mepivacaine, or tetracaine).

In a further embodiment of the third aspect, the composition further includes a skin penetration enhancer (e.g., polyacrylic acid polymer, a polysaccharide gum, isopropyl myristate, isopropyl palmitate, dimethyl sulfoxide, decyl methyl sulfoxide, dimethylalanine amide of a medium chain fatty acid, dodecyl 2-(N,N-dimethylamino) propionate, tetradecyl (N,N-dimethylamino) acetate, dodecyl (N,N-dimethylamino) acetate, decyl (N,N-dimethylamino) acetate, octyl (N,N-dimethylamino) acetate, and dodecyl (N,N-diethylamino) acetate, or salts thereof).

In another embodiment of the third aspect, the composition is formulated as a cream, a gel, a lotion, an ointment, or a liquid.

In a fourth aspect, the invention features a kit including the composition as described herein, instructions for administering the composition to a subject, and an applicator for applying the composition.

In a fifth aspect, the invention features a kit including the composition as described herein further including one or more therapeutic agents, instructions for administering the composition to a subject, and an applicator for applying the composition.

### Definitions

As used herein, the term “administration” or “administering” refers to a method of giving a dosage of a composition to a subject. The preferred method of administration may depend on a variety of factors, e.g., the components of the composition and the nature and severity of the disease, disorder, or condition.

As used herein, the phrases “an effective amount” or “an amount that is effective to treat the inflammation” refers to an amount of a composition or a compound that prevents or relieves inflammation; delays the onset of inflammation; decreases the length of a viral outbreak that results in inflammation; or diminishes the frequency or intensity of one or more symptoms associated with inflammation.

By “affected area” is meant the region of a subject that displays one or more symptoms of inflammation.

The phrase “dermatologically acceptable” means that the compositions or components thereof are suitable for use in contact with dermal tissue without undue toxicity, incompatibility, instability, allergic response, and the like.

By “subject” is meant a mammal, including, but not limited to, a human or non-human mammal.

By “treatment” is meant an approach for obtaining beneficial or desired results, such as clinical results. Beneficial or desired results can include, but are not limited to, alleviation, amelioration, or prevention of a disease, a disorder, a condition, or one or more symptoms associated with a disease, a disorder, or a condition; diminishment of extent of disease, disorder, or condition; stabilization (i.e., not worsening) of a disease, disorder, or condition; delay or slowing the progress of a disease, disorder, or condition; and amelioration or palliation of a disease, disorder, or condition. Treatment can also mean prolonging survival as compared to expected survival if not receiving treatment.

By “prevention” is meant that a prophylactic treatment is given to a subject who has or will have a disease, a disorder, a condition, or one or more symptoms associated with a disease, a disorder, or a condition.

By “palliation” of a disease, a disorder, or a condition is meant that the extent and/or undesirable clinical manifestations of the disease, disorder, or condition are lessened and/or the time course of the progression is slowed or lengthened, as compared to the extent or time course in the absence of treatment.

The recitation herein of numerical ranges by endpoints is intended to include all numbers subsumed within that range (e.g., a recitation of 1 to 5 includes 1, 1.5, 2, 2.75, 3, 3.80, 4, and 5).

As used herein, “a” or “an” means at least one or one or more unless otherwise indicated. In addition, the singular forms “a,” “an,” and “the,” include plural referents unless the context clearly dictates otherwise. Thus, for example, reference to “a composition containing a therapeutic agent” includes a mixture of two or more therapeutic agents.

Other features and advantages of the invention will be apparent from the following Detailed Description and from the claims.

### DETAILED DESCRIPTION

This invention features a method for treating inflammation of skin, including symptoms associated with inflammation. The method involves topical administration of a base composition, either alone or in combination with one or more therapeutic agents, to the affected area of a subject.

The methods and compositions of the invention can be used to treat inflammation of skin either by preventing, delaying, or relieving inflammation or by diminishing the frequency or intensity of one or more symptoms associated with inflammation. Inflammation of the skin can be caused by or associated with any number of diseases or conditions, including pruritus, viral-induced inflammation, eczema, shingles, psoriasis, atopic dermatitis, bacterial-induced inflammation, fungal-induced inflammation, burns, laceration damage, and acute injuries. Examples of viral-induced inflammation include inflammation induced by herpes simplex virus (HSV-1 and HSV-2), varicella-zoster virus, measles virus, mumps virus, human papilloma virus, and rubella virus. Examples of bacterial-induced inflammation include inflammation arising from impetigo, folliculitis, furuncles, carbuncles, cellulitis, paronychia, and hot tub folliculitis. Examples of fungal-induced inflammation include inflammation arising from onychomycosis, tinea versicolor, tinea corporis, intertrigo, and tinea pedis.

When the condition is herpes simplex virus-induced inflammation, inflammation is typically associated with the presence of cold sores or fever blisters. In particular, the methods and compositions of the invention are used to treat inflammation of cold sores. Surprisingly, as describe in more detail in the example section below, use of the inventive composition disclosed herein results in a decrease in the length of a viral outbreak from 6 days to about 2 to 3 days.

Exemplary symptoms of inflammation of skin include: erythema, blistering, edema, redness, pain, increased heat to the affected area, swelling, loss of function, decreased sensation, itching, burning, or formation of ulcers.

Base Composition and Therapeutic agents

In one aspect, this invention features a base composition comprising all natural ingredients for the prophylaxis of or cessation of inflammation and/or the induction of healing (new tissue replacement). Generally, the base composition comprises 70% to 95% (w/w) of one or more waxes, 5% to 10% (w/w) of essential extracts, 0.1% to 1.0% (w/w) of a thickener, and 0.1% to 0.5% (w/w) of an antioxidant. Optionally, the base composition can include 5% to 10% (w/w) of an herbal infused oil.

A wax is a lipophilic fatty compound that is solid or semi-solid at room temperature (25° C.). Examples of waxes include any dermatologically acceptable wax, including beeswax, carnauba wax, lanolin, Chinese insect waxes, rice wax, candelilla wax, ouricury wax, cork fiber wax, sugar cane wax, Japan wax, sumach wax, montan wax, microcrystalline waxes, paraffin waxes, ozokerites, ceresin wax, lignite wax, polyethylene waxes, fatty acid esters of glycerides, and hydrogenated animal or plant oils (e.g., hydrogenated jojoba oil, hydrogenated sunflower oil, hydrogenated castor oil, hydrogenated coconut oil and hydrogenated lanolin oil). Preferred waxes are beeswax, carnauba wax, and lanolin.

The herbal infused oil can be any dermatologically acceptable oil that has been infused with one or more of the following herbs: lemon balm (Melissa officinalis), lavender, lemon grass, lemon verbena, mint, calendula flowers (Calendula officinalis), chamomile flowers, eucalyptus, sage, green tea gunpowder (Camellia sinensis), white tea powder, and green rooibos (Aspalatus linearis). Dermatologically acceptable oils include, but are not limited to, oil obtained from plants such as rapeseed (Brassica spp.), soybean (Glycine max), oil palm (Elaeis guineeis), coconut (Cocus nucifera), castor (Ricinus communis), safflower (Carthamus tinctorius), mustard (Brassica spp. and Sinapis alba), coriander (Coriandrum sativum) linseed/flax (Linum usitatissimum), thale cress (Arabidopsis thaliana), and maize (Zea mays). A preferred embodiment of the herbal infused oil is coconut oil infused with lemon balm (Melissa officinalis), calendula flowers (Calendula officinalis), green tea gunpowder (Camellia sinensis), and green rooibos (Aspalatus linearis).

Essential extracts include those oils or compounds extracted or obtained from plants and seeds or artificially obtained substitutes. Exemplary essential extracts include those obtained from rosemary, basil, ginger, sweet orange, Geranium Egypt, peppermint, Tea Tree, vanilla, stevia, hempseed, sweet almond, and castor seed.

In one embodiment, the base composition comprises 70% to 95% (w/w) of beeswax, carnauba wax, and lanolin; 5% to 10% (w/w) of one or more essential extracts; 0.1% to 1.0% (w/w) of caprylic/capric triglycerides; and 0.1% to 0.5% (w/w) of tocopherol acetate.

In another embodiment, the base composition comprises 70% to 95% (w/w) of beeswax, carnauba wax, and lanolin; 5% to 10% (w/w) of one or more essential extracts; 5% to 10% (w/w) of an herbal infused oil; 0.1% to 1.0% (w/w) of caprylic/capric triglycerides; and 0.1% to 0.5% (w/w) of tocopherol acetate.

In yet another embodiment, the base composition comprises 70% to 95% (w/w) of beeswax, carnauba wax, and lanolin; 5% to 10% (w/w) of essential extracts of rosemary oil (Rosmarinus officinalis), basil oil (Ocimum basilicum), ginger oil (Zingiber officinale Roscoe), sweet orange oil (Citrus sinensis), Geranium Egypt oil (Pelargonium graveolens), lemon oil (Citrus limonum), peppermint oil (Mentha piperita), Tea Tree oil (Melaleuca alternifolia), vanilla infused oil, stevia (Eupatorium rebaudianum), sweet almond oil, castor seed oil, hydrogenated castor oil, and hempseed oil; 5% to 10% (w/w) of a coconut oil; 0.1% to 1.0% (w/w) of caprylic/capric triglycerides; and 0.1% to 0.5% (w/w) of tocopherol acetate.

In a further embodiment, the base composition comprises 73% beeswax; 22% lip balm base, which includes 30% to 90% of a combination of beeswax, castor seed oil, hydrogenated castor oil, and carnauba wax, 3% to 10% sweet almond oil, 1% to 3% caprylic/capric triglycerides, 0.3% to 1% lanolin, 0.3% to 1% tocopherol acetate, and <0.1% hempseed oil; and about 5% of the following essential extracts: rosemary, 0.3%; basil, 0.3%; ginger, 0.3%; sweet orange, 1.0%; Geranium Egypt, 0.3%; peppermint, 0.9%; Tea Tree, 0.3%; vanilla infused oil, 0.7%; and stevia, 0.3%.

In another aspect, the methods and compositions of the invention utilizes the base composition in combination with one or more therapeutic agents. Suitable therapeutic agents in the compositions and methods of the invention generally include those that will act locally to prevent or relieve inflammation. For example, the compositions may contain one or more therapeutic agents that provide an antihistaminic effect. The antihistaminic effect may be provided in any number of ways, such as by H-1 receptor antagonism, by preventing mast cell degranulation, or by preventing the release of histamine contained in mast cells.

Examples of therapeutic agents that may be used in the inventive compositions include, but are not limited to, antibacterial agents, antifungal agents, antihistamines, antiinflammatory agents, antiviral agents, ion channel blocking agents, and opioids.

For those embodiments in which the composition is applied topically to the subject, the therapeutic agents used in the composition should have appropriate properties for topical administration. For example, suitable therapeutic agents for topical formulations include those that will act locally and upon absorption will be diluted into the large blood volume of the vascular space; or that will produce no adverse events. The composition should also not induce skin irritation or exhibit photosensitivity to the skin.

Exemplary antibacterial agents include, but are not limited to, demeclocycline, chlortetracycline, oxytetracycline, tetracycline, chloramphenicol, neomycin, gentamicin, amikacin, clindamycin, nadifloxacin, streptogramin, virginiamycin, rifamycin, rifaximin, fusidic acid, bacitracin, tyrothricin, or mupirocin.

Exemplary antifungal agents include, but are not limited to, terbinafine hydrochloride, clotrimazole, ketoconazole, nystatin, natamycin, hachimycin, pecilocin, mepartricin, pyrrolnitrin, griseofulvin, miconazole, econazole, clomidazole, isoconazole, tiabendazole, tioconazole, sulconazole, bifonazole, oxiconazole, fenticonazole, omoconazole, sertaconazole, fluconazole, flutrimazole, enilconazole, bromochlorosalicylanilidc, methylrosaniline, tribromometacresol, undecylenic acid, polynoxylin, 2-(4-chlorphenoxy)-ethanol, chlorphenesin, ticlatone, sulbentine, ethyl hydroxybenzoate, haloprogin, salicylic acid, selenium sulfide, ciclopirox, amorolfine, dimazole, tolnaftate, tolciclate, flucytosine, naftifine, butenafine, undecylenic acid, bronopol, or bensuldazic acid.

Exemplary antihistamines (e.g., H-1 receptor antagonists) include, but are not limited to a tricyclic antidepressant with H-1 receptor antagonism and/or sodium channel blocking activity (e.g., doxepin, imipramine, trimipramine, amitriptyline, clomipramine, amoxapine, desipramine, lofepramine, maprotiline, nortriptyline, mirtazapine, opipramol, or protriptyline); an cthanolamine agent (e.g., carbinoxamine, clemastine, or diphenhydramine); an ethylenediamine agent (e.g., pyrilamine or tripelennamine); an alkylamine agent (e.g. triprolidine, acrivastine, chlorpheniramine, or brompheniramine); a piperazine agent (e.g. hydroxyzine, cyclizine, or meclizine); a phenothiazine agent (e.g., promethazine or chlorpromazine); or a piperidine agent (e.g., cyproheptadine or phenindamine), as well as promazine and chlorpromazine. Most preferred among the antihistamines for formulation with the base composition are doxepin, amitriptyline, triprolidine, acrivastine, and diphenhydramine.

Exemplary antiinflammatory agents include, but are not limited to, cyclooxygenase (COX) inhibitors and non-steroidal antiinflammatory drugs (NSAIDs). Examples of antiinflammatory compounds include aspirin, diclofenac, ibuprofen, including a racemic mixture or an enantiomer thereof; ketoprofen, including a racemic mixture or an enantiomer thereof; or naproxen.

Exemplary antiviral agents include, but are not limited to, acyclovir, cidofovir, docosanol, famciclovir, foscarnet, fomivirsen, ganciclovir, idoxuridine, penciclovir, peramivir, trifluridine, valacyclovir, vidarabine, lamivudine, or ribavirin.

Exemplary ion channel blocking agents include all classes of sodium channel blocking agents, such as benzocaine, bupivacaine, lidocaine, etidocaine, mepivacaine, pramoxine (also known as pramocaine), prilocaine, procaine, proparacaine, ropivacaine, or tetracaine. Other ion channel blocking agents include phenytoin and derivatives thereof, as well as acid sensitive ion channel blocking agents, such as amiloride and derivatives thereof.

Exemplary opioids include morphine, codeine, meperidine, and oxycodone.

Combinations of two or more therapeutic agents can be administered to a subject to treat inflammation of skin. Exemplary combinations include a combination of two antihistamines from different chemical classes, such as a tricyclic antidepressant with an ethanolamine agent (e.g., doxepin and diphenhydramine) or a tricyclic depressant with an alkylamine agent (e.g., doxepin and triprolidine or acrivastine); a combination of an antihistamine and an antiinflammatory (e.g. doxepin and ketoprofen); a combination of an antihistamine and an antiviral agent (e.g., doxepin and one or more antivirals such as acyclovir or valacyclovir); and a combination of an antihistamine and an ion channel blocking agent (e.g. doxepin and lidocaine, or doxepin and a mixture of ion channel blocking agents with short- and intermediate-term anesthetic action, such as the combination of benzocaine and tetracaine).

### Dosage, Formulation, and Administration

The compositions of the invention may conveniently be administered in unit dosage form and may be prepared by any of the methods well-known in the pharmaceutical art, for example, as described in Remington: The Science and Practice of Pharmacy (20th ed., ed. A. R. Gennaro, 2000, Lippincott Williams & Wilkins). The concentration of one or more of the components of the base composition or one or more therapeutic agents in the formulation will vary depending upon a number of factors, including the dosage of the one or more therapeutic agents to be administered, and the route of administration.

The therapeutic agents may be optionally administered in the form of the chemical base or as a pharmaceutically acceptable salt thereof, such as a non-toxic acid addition salts or metal complexes that are commonly used in the pharmaceutical industry. Examples of acid addition salts include organic acids such as acetic, lactic, pamoic, maleic, citric, malic, ascorbic, succinic, benzoic, palmitic, suberic, salicylic, tartaric, methanesulfonic, toluenesulfonic, or trifluoroacetic acids or the like; polymeric acids such as tannic acid, carboxymethyl cellulose, or the like; and inorganic acid such as hydrochloric acid, hydrobromic acid, sulfuric acid phosphoric acid, or the like. Metal complexes include zinc, iron, and the like.

The therapeutic agents may also be derivatives of any compound described herein. Derivatives of compounds are well known in the art. Derivatives of compounds include modifications within the backbone of the molecule and modifications to the pendant groups of the molecule. Modifications within the backbone of the molecule include use of substitutions selected from the following groups: O, N, and S; or C—C, C═C, and C═C. Modifications to the pendant groups include use of substitutions selected from the following groups: H and alkyl; hydroxyl and sulfhydryl; pyridyl, pyranyl, and thiopyranyl; piperidyl, tetrahydropyranyl, and thianyl; or piperazinyl, morpholinyl, dithianyl, and dioxanyl.

The base composition alone or in combination with one or more therapeutic agents can be prepared in any useful method. In general, the base composition is prepared with the lip balm base, the herbal infused oil, and the essential extracts, and then maintained in a liquid state with mild heating at 50° C. In one embodiment, the base composition is used without additional therapeutic agents. The lip balm base is mixed with the herbal infused oil and the essential extracts, where the resulting base composition in the liquid state is poured into tubes, tins, droptainers or other dispensing devices. The base composition is then allowed to cool.

In another embodiment, the base composition is prepared in combination with one or more therapeutic agents. One or more therapeutic agents are weighed out and placed in a solvent or solvent mixture using mild conditions, such as by sonicating or heating in the presence of ethanol, 1% dimethylsulfoxide, or polyethylene glycol. Once the one or more therapeutic agents are in solution, they are added to the previously prepared base composition in a liquid state with constant stirring. Stirring under these conditions continues for minimally 30 minutes and then the composition is poured into tubes, tins, droptainers, or other dispensing devices, and allowed to cool.

The base composition can be prepared with any solvent system, such as those Generally Regarded as Safe (GRAS) by the U.S. Food & Drug Administration (FDA). GARS solvent systems include many short chain hydrocarbons, such as butane, propane, n-butane, or a mixture thereof, as the delivery vehicle, which are approved by the FDA for topical use.

Optimization of the appropriate dosages can readily be made by the skilled practitioner in light of the pharmacokinetics of the base composition or one or more therapeutic agents used in the composition. Factors to be considered in setting dosages include the compounds specific activity; the severity of the condition or symptoms of the subject; the age, condition, body weight, sex, and diet of the subject; the use (or not) of concomitant therapies; and other clinical factors.

Administration may be one or multiple times daily, weekly (or at some other multiple day interval) or on an intermittent schedule, with that cycle repeated a given number of times (e.g., 2-10 cycles) or indefinitely. Alternatively, the compositions may be administered as symptoms occur.

The compositions are typically administered daily. The composition can be used ad libitum or used as a prophylactic. Most commonly, this composition can be administered daily, such as one, two, or three times daily. In one embodiment, the composition comprises the base composition. In another embodiment, the composition comprises between 0.1% to 30% (w/w) of one or more therapeutic agents (e.g., 0.1%-1%, 0.5%-2%, 1%-5%, 1%-10%, 5%-l0%, 5%-20%, 10%-20%, 10%-25%, or 15%-30% (w/w)). Preferred dosages include 1% to 10% (w/w) of one or more therapeutic agents in the base composition, or 10% to 25% (w/w) of two or more therapeutic agents in the base composition.

The compositions can be formulated using any dermatologically acceptable carrier. Exemplary carriers include a solid carrier, such as alumina, clay, microcrystalline cellulose, silica, or talc; and/or a liquid carrier, such as an alcohol, a glycol, or a water-alcohol/glycol blend. The compounds may also be administered in liposomal formulations that allow compounds to enter the skin. Such liposomal formulations are described in U.S. Pat. Nos. 5,169,637; 5,000,958; 5,049,388; 4,975,282; 5,194,266; 5,023,087; 5,688,525; 5,874,104; 5,409,704; 5,552,155; 5,356,633; 5,032,582; 4,994,213; and PCT Publication No. WO 96/40061. Examples of other appropriate vehicles are described in U.S. Pat. No. 4,877,805, U.S. 4,980,378, U.S. 5,082,866, U.S. 6,118,020 and EP Publication No. 0586106A1. Suitable vehicles of the invention may also include mineral oil, petrolatum, polydecene, stearic acid, isopropyl myristate, polyoxyl 40 stearate, stearyl alcohol, or vegetable oil.

The compositions can be provided in any useful form. For example, the compositions of the invention may be formulated as solutions, emulsions (including microemulsions), suspensions, creams, foams, lotions, gels, powders, balm, or other typical solid, semi-solid, or liquid compositions used for application to the skin or other tissues where the compositions may be used. Such compositions may contain other ingredients typically used in such products, such as colorants, fragrances, thickeners, antimicrobials, solvents, surfactants, detergents, gelling agents, antioxidants, fillers, dyestuffs, viscosity-controlling agents, preservatives, humectants, emollients (e.g., natural or synthetic oils, hydrocarbon oils, waxes, or silicones), hydration agents, chelating agents, demulcents, solubilizing excipients, adjuvants, dispersants, skin penetration enhancers, plasticizing agents, preservatives, stabilizers, demulsifiers, wetting agents, sunscreens, emulsifiers, moisturizers, astringents, deodorants, and optionally including anesthetics, anti-itch actives, botanical extracts, conditioning agents, darkening or lightening agents, glitter, humectants, mica, minerals. polyphenols, silicones or derivatives thereof, sunblocks, vitamins, and phytomedicinals. Exemplary antioxidants include ascorbic acid, tocopherols (e.g., α-, β-, γ-, δ-tocopherols, and derivatives thereof, such as tocopherol acetate), lipoic acid, sodium bisulfite, potassium bisulfite, ascorbyl palmitate, butylated hydroxyanisole, butylated hydroxy toluene, potassium metabisulfite, sodium metabisulfite, sodium thiosulfate, thiourea, and the like. Preferred antioxidants include tocopherols, in particular, tocopherol acetate.

Exemplary thickeners include xanthan gum, a fatty acid, including triglycerides, a fatty acid salt or ester, a fatty alcohol, a modified cellulose, a modified mineral material, or a synthetic polymer. A preferred thickener is caprylic/capric triglyceride.

The compositions can also include other like ingredients to provide additional benefits and improve the feel and/or appearance of the topical formulation. To the standard base composition, various skin penetration enhancers may be added such as deoxycholate, palmitate, or dimethylalanineamides of medium chain fatty acids, as described in U.S. Pat. Nos. 4,877,805, 4,980,378, 5,082,866, and 6,118,020, which are incorporated herein by reference.

In particular, compositions for topical application can further include a skin penetration enhancer, such as those described in “Percutaneous Penetration enhancers”, (eds. Smith E W and Maibach H I. CRC Press 1995). Exemplary skin penetration enhancers include alkyl (N,N-disubstituted amino alkanoate) esters, such as dodecyl 2-(N,N-dimethylamino) propionate (DDAIP), which is described in patent U.S. Pat. Nos. 6,083,996 and 6,118,020, which are both incorporated herein by reference; a water-dispersible acid polymer, such as a polyacrylic acid polymer, a carbomer (e.g., Carbopol™ or Carbopol 940P™, available from B. F. Goodrich Company (Akron, Ohio)), copolymers of polyacrylic acid (e.g., Pemulen™ from B. F. Goodrich Company or Polycarbophil™ from A. H. Robbins, Richmond, Va.; a polysaccharide gum, such as agar gum, alginate, carrageenan gum, ghatti gum, karaya gum, kadaya gum, rhamsan gum, xanthan gum, and galactomannan gum (e.g., guar gum, carob gum, and locust bean gum), as well as other gums known in the art (see for instance, Industrial Gums: Polysaccharides & Their Derivatives, Whistler R. L., BeMiller J. N. (eds.), 3rd Ed. Academic Press (1992) and Davidson, R. L., Handbook of Water-Soluble Gums & Resins, McGraw-Hill, Inc., N.Y. (1980)); or combinations thereof.

Other suitable polymeric skin penetration enhancers are cellulose derivatives, such as ethyl cellulose, methyl cellulose, hydroxypropyl cellulose. Additionally, known transdermal skin penetration enhancers can also be added, if desired. Illustrative are dimethyl sulfoxide (DMSO) and dimethyl acetamide (DMA), 2-pyrrolidone, N,N-diethyl-m-toluamide (DEET), 1-dodecylazacycloheptane-2-one (Azone™, a registered trademark of Nelson Research), N,N-dimethylformamide, N-methyl-2-pyrrolidone, calcium thioglycolate and other enhancers such as dioxolanes, cyclic ketones, and their derivatives and so on.

Also illustrative are a group of biodegradable skin penetration enhancer, which are alkyl N,N-2-(disubstituted amino) alkanoates as described in U.S. Pat. No. 4,980,378 and U.S. Pat. No. 5,082,866, which are both incorporated herein by reference, including tetradecyl (N,N-dimethylamino) acetate, dodecyl (N,N-dimethylamino) acetate, decyl (N,N-dimethylamino) acetate, octyl (N,N-dimethylamino) acetate, and dodecyl (N,N-diethylamino) acetate.

Particularly preferred skin penetration enhancers include isopropyl myristate; isopropyl palmitate; dimethyl sulfoxide; decyl methyl sulfoxide; dimethylalanine amide of a medium chain fatty acid; dodecyl 2-(N,N-dimethylamino) propionate or salts thereof, such as its organic (e.g., hydrochloric, hydrobromic, sulfuric, phosphoric, and nitric acid addition salts) and inorganic salts (e.g., acetic, benzoic, salicylic, glycolic, succinic, nicotinic, tartaric, maleic, malic, pamoic, methanesulfonic, cyclohexanesulfamic, picric, and lactic acid addition salts), as described in U.S. Pat. No. 6,118,020; and alkyl 2-(N,N-disubstituted amino)-alkanoates, as described in U.S. Pat. No. 4,980,378 and U.S. Pat. No. 5,082,866.

When included in the composition, the skin penetration enhancer in this composition by weight would be in the range of 0.5% to 10% (w/w). The most preferred range would be between 1.0% and 5% (w/w). In another embodiment, the skin penetration enhancer comprises between 0.5% -1%, 1%-2%, 2%-3%, 3%-4%, or 4%-5%, (w/w) of the composition.

The compositions can be administered in any number of ways. For example, the compositions in liquid form can be applied from absorbent pads; used to impregnate bandages and other dressings; or sprayed directly onto the affected area of the subject. In another example, the composition in solid form, including semi-solid form, can be applied from a tube; or the composition in liquid form or solid form is applied directly onto the affected area of the subject. In yet another example, the composition in liquid form or solid form can be applied by using an applicator (e.g., a stick or a swab) to spread the composition onto the affected area. The composition may also be applied to the skin under occlusive dressing in a dermal delivery system (e.g., a transdei anal patch).

In a preferred embodiment, the compositions are intended for topical use in form of a chap stick; a lotion in a tin or a tube; or a liquid, where a liquid applicator such as a swab may be used to administer the active formulation. Standard formulations that are used in the art of preparing topical agents are incorporated herein. These formulations include those of varying viscosity (e.g., liquid, semi-solid, solid, and emulsion forms), including lotions and chap stick.

Administration of compounds in controlled release formulations may be useful where the one or more compounds have (i) a narrow therapeutic index (e.g., the difference between the plasma concentration leading to harmful side effects or toxic reactions and the plasma concentration leading to a therapeutic effect is small); (ii) a narrow slow absorption rate by or through the epithelium and/or dermis; or (iii) a short biological half-life, so that frequent dosing during a day is required in order to sustain a therapeutic level.

Many strategies can be pursued to obtain controlled release in which the rate of release outweighs the rate of metabolism of the therapeutic compound. For example, controlled release can be obtained by the appropriate selection of formulation parameters and ingredients, including, e.g., appropriate controlled release compositions and coatings. Examples include oil solutions, suspensions, emulsions, microcapsules, microspheres, nanoparticles, patches, and liposomes.

Further features and advantages of this invention are further illustrated by the following examples, which arc in no way intended to be limiting thereof.

### EXAMPLES

### Example 1

### Preparation of Herbal Infused Oil

One gallon of coconut oil was heated until it was completely in a liquid state. The following herbs were weighed (2 oz. each) and combined together: Lemon Balm (Melissa officinalis); Calendula Flowers (Calendula officinalis); Green Tea Gunpowder (Camellia sinensis); and Green Rooibos (Aspalatus linearis). The herbs were added to liquid coconut oil with constant stirring. Heat was maintained between 110° F. and 140° F. Do not exceed heat above 140° F., as this will cause the herbs to burn. Preferably, the heat should be maintained between 110° F. and 120° F. for 3 hours (the extraction period). After this extraction period, liquid mixture was strained using a stainless steel colander. A steel paddle was used to press the residue in order to squeeze out the remaining oils. The resulting liquid was strained again using a fine mesh filter to eliminate all remaining particulate matter, which produced a clear, herbal infused oil. This oil was used in the preparation of the lip balm base (Example 2).

### Example 2

### Preparation of the Base Composition

Lip balm base (22 oz., 3¾ cups, 625 grams, from New Directions Aromatics, Inc.) was heated until in a liquid form. Granulated beeswax (72 oz., 9 cups, 2045 grams, CandleChem Co.) was heated until in liquid form. The lip balm base and granulated beeswx was combined with stirring. This solution was added to the previous prepared herbal infused oil (as in Example 1). Heat was maintained at a temperature between 110° F. and 130° F. to insure that all elements of this mixture remain in liquid form as a homogeneous mixture.

The following essential extracts were separately pipetted and mixed together: 6 ml of Rosemary oil from Soma Therapy, 6 ml of basil oil from Soma Therapy, 6 ml of Ginger from Soma Therapy, 29 ml of Sweet Orange from Dreaming Earth Botanicals, 6 ml of Geranium Egypt from Dreaming Earth Botanicals, 18 ml of Lemon Extract from Soma Therapy, 23 ml of Peppermint from THE CHEMISTRY, 6 ml of Tea Tree from Mountain Rose Herbs, 21 ml of vanilla extract from Magestic Mountain Sage, and 11 grams of stevia (multiple sources). The essentials extracts were stirred into the all liquid and homogeneous mixture of beeswax and lip balm base containing the previously prepared herbal infused oil. This base composition is an all natural formulation and can now be poured into dispensing tubes, tins, etc., or used as a base to add therapeutic agents.

### Example 3

### Preparation of the Base Composition with Therapeutic Agents

To prepare 1 liter of base composition with therapeutic agents, 10 to 100 grams or 1-10% of each therapeutic agent were weighed. The therapeutic agents were dissolved in an appropriate all GRAS organic solvent system. If more than one therapeutic agent is used, this step was repeated. One or more therapeutic agents were added to the liquid form of the base composition (as prepared in Example 2) and stirred to make a homogeneous mixture. The contents were poured into an appropriate dispensing container (e.g., chap stick tubes, tins, droptainers, etc.). This general procedure could be used to make compositions having various strengths of therapeutic agents, such as 5% strength by weighing out 50 grams of therapeutic agents and adding to a liter of natural base. This procedure allows for combinations of therapeutic agents at varying strengths, such as 10% (w/w) of an antiviral agent and 5% (w/w) of an antihistamine.

### Example 4

### Treatment of Patients with Base Composition

The study included six patients with a history of constant, recurring cold sores. The prophylactic use of the base composition (lip balm base with the addition of the herbal infused oil and essential extracts) resulted in no re-occurrence of cold sores over a period of 3 months.

### Example 5

### Treatment of Patients with Base Composition and Doxepin

The study included 2 female and 2 male patients with active cold sores, which exhibited edema, blistering, and itching. Doxepin, an antihistamine, was added to the base composition at a strength of 5% (w/w). When this formulation was added to an active cold sore, the cold sores dried up (scabbed over) in less than 36 hours for the patients. No skin irritation or photosensitization was noted.

All publications, patent applications, and patents mentioned in this specification are herein incorporated by reference.

Various modifications and variations of the described method and system of the invention will be apparent to those skilled in the art without departing from the scope and spirit of the invention. Although the invention has been described in connection with specific desired embodiments, it should be understood that the invention as claimed should not be unduly limited to such specific embodiments. Indeed, various modifications of the described modes for carrying out the invention that are obvious to those skilled in the fields of medicine, pharmacology, or related fields are intended to be within the scope of the invention.

What is claimed is:

1. A method of treating a subject suffering from a herpes simplex virus-induced inflammation, the method comprising topically applying to an affected area of the subject a composition comprising an effective amount of an antihistamine.

2. The method of claim 1, wherein the inflammation is a Herpes Simplex Virus-1 (HSV-1)-induced inflammation.

3. The method of claim 1, wherein the inflammation is a Herpes Simplex Virus-2 (HSV-2)-induced inflammation.

4. The method of claim 1, wherein the antihistamine is selected from the group consisting of doxepin, amitriptyline, triprolidine, acrivastine, and diphenhydramine.

5. The method of claim 1, wherein the composition further comprises an ion channel blocking agent and an antiviral agent.

6. A method of treating inflammation of skin in a subject, the method comprising topically administering to an affected area of the subject a base composition in an amount that is effective to treat the inflammation, wherein the base composition comprises 70% to 95% (w/w) of one or more waxes, 5% to 10% (w/w) of one or more essential extracts, 0.1% to 1.0% (w/w) of a thickener, and 0.1% to 0.5% (w/w) of an antioxidant.

7. The method of claim 6, wherein the inflammation is associated with one or more of pruritus, viral-induced inflammation, eczema, shingles, psoriasis, atopic dermatitis, bacterial-induced inflammation, fungal-induced inflammation, burns, laceration damage, and acute injuries.

8. The method of claim 7, wherein the inflammation is viral-induced inflammation.

9. The method of claim 8, wherein the viral-induced inflammation is associated with a cold sore.

10. The method of claim 6, wherein the one or more waxes are selected from the group consisting of beeswax, carnauba wax, and lanolin.

11. The method of claim 6, wherein the one or more essential extracts are selected from the group consisting of rosemary oil (Rosmarinus officinalis), basil oil (Ocimum basilicum), ginger oil (Zingiber officinale Roscoe), sweet orange oil (Citrus sinensis), Geranium Egypt oil (Pelargonium graveolens), lemon oil (Citrus limonum), peppermint oil (Mentha piperita), Tea Tree oil (Melaleuca alternifolia), vanilla infused oil, stevia (Eupatorium rebaudianum), sweet almond oil, castor seed oil, hydrogenated castor oil, and hempseed oil.

12. The method of claim 6, wherein the thickener is a caprylic/capric triglyceride.

13. The method of claim 6, wherein the antioxidant is tocoperol or a derivative thereof

14. The method of claim 6, wherein the base composition further comprises 5% to 10% (w/w) of an herbal infused oil.

15. The method of claim 14, wherein the herbal infused oil is coconut oil infused with lemon balm (Melissa officinalis), calendula flowers (Calendula officinalis), green tea gunpowder (Camellia sinensis), and green rooibos (Aspalatus linearis).

16. The method of claim 6, wherein the base composition further comprises one or more therapeutic agents selected from the group consisting of an antibacterial agent, an antifungal agent, an antihistamine, an antiinflammatory agent, an antiviral agent, an ion channel blocking agent, and an opioid.

17. The method of claim 16, wherein the therapeutic agent is the antibacterial agent and the antibacterial agent is selected from the group consisting of demeclocycline, chlortetracycline, oxytetracycline, tetracycline, chloramphenicol, neomycin, gentamicin, amikacin, clindamycin, nadifloxacin, streptogramin, virginiamycin, rifamycin, rifaximin, fusidic acid, bacitracin, tyrothricin, and mupirocin.

18. The method of claim 16, wherein the therapeutic agent is the antifungal agent and the antifungal agent is selected from the group consisting of terbinafine hydrochloride, clotrimazole, ketoconazole, nystatin, natamycin, hachimycin, pecilocin, mepartricin, pyrrolnitrin, griseofulvin, miconazole, econazole, clomidazole, isoconazole, tiabendazole, tioconazole, sulconazole, bifonazole, oxiconazole, fenticonazole, omoconazole, sertaconazole, fluconazole, flutrimazole, enilconazole, bromochlorosalicylanilide, methylrosaniline, tribromometacresol, undecylenic acid, polynoxylin, 2-(4-chlorphenoxy)-ethanol, chlorphenesin, ticlatone, sulbentine, ethyl hydroxybenzoate, haloprogin, salicylic acid, selenium sulfide, ciclopirox, amorolfine, dimazole, tolnaftate, tolciclate, flucytosine, naftifine, butenafine, undecylenic acid, bronopol, and bensuldazic acid.

19. The method of claim 16, wherein the therapeutic agent is the antihistamine and the antihistamine is selected from the group consisting of a tricyclic antidepressant, an ethanolamine agent, an ethylenediamine agent, an alkylamine agent, a piperazine agent, a phenothiazine agent, and a piperidine agent.

20. The method of claim 19, wherein the antihistamine is a tricyclic antidepressant and the tricyclic antidepressant is doxepin or amitriptyline or a pharmaceutically acceptable salt thereof.

21. The method of claim 19, wherein the antihistamine is an ethanolamine agent and the ethanolamine agent is diphenhydramine.

22. The method of claim 19, wherein the antihistamine is the alkylamine agent and the alkylamine agent is triprolidine, acrivastine, or chlorpheniramine.

23. The method of claim 19, wherein the antihistamine is the phenothiazine agent and the phenothiazine agent is promethazine or chlorpromazine.

24. The method of claim 19, wherein the antihistamine is the piperidine agent and the piperidine agent is cyproheptadine.

25. The method of claim 16, wherein the therapeutic agent is the antiinflammatory agent and the antiinflammatory agent is selected from the group consisting of aspirin, diclofenac, ibuprofen, ketoprofen, and naproxen.

26. The method of claim 16, wherein the therapeutic agent is the antiviral agent and the antiviral agent is selected from the group consisting of acyclovir, cidofovir, docosanol, famciclovir, foscarnet, fomivirsen, ganciclovir, idoxuridine, penciclovir, peramivir, trifluridine, valacyclovir, vidarabine, lamivudine, and ribavirin.

27. The method of claim 16, wherein the therapeutic agent is the ion channel blocking agent and the ion channel blocking agent is a sodium channel blocking agent or an acid sensitive ion channel blocking agent.

28. The method of claim 27, wherein the ion channel blocking agent is the sodium channel blocking agent and the sodium channel blocking agent is selected from the group consisting of benzocaine, bupivacaine, lidocaine, etidocaine, mepivacaine, pramoxine, prilocaine, procaine, proparacaine, ropivacaine, and tetracaine.

29. The method of claim 27, wherein the ion channel blocking agent is the acid sensitive ion channel blocking agent and the acid sensitive ion channel blocking agent is amiloride or derivatives or pharmaceutically acceptable salts thereof.

30. The method of claim 16, wherein the therapeutic agent is the opioid and the opioid is selected from the group consisting of morphine, codeine, meperidine, and oxycodone.

31. The method of claim 16, wherein the one or more therapeutic agents are one or more antihistamines selected from the group consisting of a tricyclic antidepressant, an ethanolamine agent, an ethylenediamine agent, an alkylamine agent, a piperazine agent, a phenothiazine agent, and a piperidine agent.

32. The method of claim 31, wherein the one or more therapeutic agents are the tricyclic antidepressant and the ethanolamine agent.

33. The method of claim 32, wherein the tricyclic antidepressant is doxepin or a pharmaceutically acceptable salt thereof and the ethanolamine agent is diphenhydramine.

34. The method of claim 31, wherein the one or more therapeutic agents are the tricyclic antidepressant and the alkylamine agent.

35. The method of claim 34, wherein the tricyclic antidepressant is doxepin or a pharmaceutically acceptable salt thereof and the alkylamine agent is triprolidine or acrivastine.

36. The method of claim 16, wherein the one or more therapeutic agents comprise one or more antihistamines selected from the group consisting of a tricyclic antidepressant, an ethanolamine agent, an ethylenediamine agent, an alkylamine agent, a piperazine agent, a phenothiazine agent, and a piperidine agent; and one or more antiinflammatory agents.

37. The method of claim 36, wherein the antihistamine is doxepin or a pharmaceutically acceptable salt thereof and the antiinflammatory agent is ketoprofen.

38. The method of claim 16, wherein the one or more therapeutic agents comprise one or more antihistamines selected from the group consisting of a tricyclic antidepressant, an ethanolamine agent, an ethylenediamine agent, an alkylamine agent, a piperazine agent, a phenothiazine agent, and a piperidine agent; and one or more antiviral agents.

39. The method of claim 38, wherein the antihistamine is doxepin or a pharmaceutically acceptable salt thereof and the one or more antiviral agents are selected from the group consisting of acyclovir and valacyclovir.

40. The method of claim 16, wherein the one or more therapeutic agents comprise one or more antihistamines selected from the group consisting of a tricyclic antidepressant, an ethanolamine agent, an ethylenediamine agent, an alkylamine agent, a piperazine agent, a phenothiazine agent, and a piperidine agent; and one or more ion channel blocking agents selected from the group consisting of a sodium channel blocking agent and an acid sensitive ion channel blocking agent.

41. The method of claim 40, wherein the antihistamine is doxepin or a pharmaceutically acceptable salt thereof and the one or more ion channel blocking agents are selected from the group consisting of lidocaine, benzocaine, and tetracaine.

42. The method of claim 16, wherein the one or more therapeutic agents comprise one or more antihistamines selected from the group consisting of a tricyclic antidepressant, an ethanolamine agent, an ethylenediamine agent, an alkylamine agent, a piperazine agent, a phenothiazine agent, and a piperidine agent; one or more ion channel blocking agents selected from the group consisting of a sodium channel blocking agent and an acid sensitive ion channel blocking agent; and one or more antiviral agents selected from the group consisting of acyclovir, cidofovir, docosanol, famciclovir, foscarnet, fomivirsen, ganciclovir, idoxuridine, penciclovir, peramivir, trifluridine, valacyclovir, vidarabine, lamivudine, and ribavirin.

43. The method of claim 16, wherein the base composition comprises 0.1% to 30% (w/w) of one or more therapeutic agents.

44. The method of claim 43, wherein the base composition comprises 1% to 10% (w/w) of one therapeutic agent.

45. The method of claim 43, wherein the base composition comprises 10% to 25% (w/w) of two or more therapeutic agents.

46. The method of claim 16, wherein the base composition comprises 1% to 10% (w/w) of doxepin or a pharmaceutically acceptable salt thereof.

47. The method of claim 16, wherein the base composition comprises 1% to 10% (w/w) of doxepin or a pharmaceutically acceptable salt thereof and 1% to 10% (w/w) of acyclovir or valacyclovir.

48. A composition formulated for topical administration comprising a base composition, wherein the base composition comprises 70% to 95% (w/w) of one or more waxes, 5% to 10% (w/w) of one or more essential extracts, 0.1% to 1.0% (w/w) of a thickener, and 0.1% to 0.5% (w/w) of an antioxidant.

49. The composition of claim 48, wherein the base composition comprises 70% to 95% (w/w) of beeswax, carnauba wax, and lanolin, 5% to 10% (w/w) of one or more essential extracts, 0.1% to 1.0% (w/w) of caprylic/capric triglycerides, and 0.1% to 0.5% (w/w) of tocopherol acetate.

50. The composition of claim 48, wherein the one or more essential extracts are selected from the group consisting of rosemary oil (Rosmarinus officinalis), basil oil (Ocimum basilicum), ginger oil (Zingiber officinale Roscoe), sweet orange oil (Citrus sinensis), Geranium Egypt oil (Pelargonium graveolens), lemon oil (Citrus limonum), peppermint oil (Mentha piperita), Tea Tree oil (Melaleuca alternifolia), vanilla infused oil, stevia (Eupatorium rebaudianum), sweet almond oil, castor seed oil, hydrogenated castor oil, and hempseed oil.

51. The composition of claim 48, further comprising 5% to 10% (w/w) of an herbal infused oil.

52. The composition of claim 51, wherein the herbal infused oil is coconut oil infused with lemon balm (Melissa officinalis), calendula flowers (Calendula officinalis), green tea gunpowder (Camellia sinensis), and green rooibos (Aspalatus linearis).

53. The composition of claim 48, further comprising one or more therapeutic agents selected from the group consisting of an antibacterial agent, an antifungal agent, an antihistamine, an antiinflammatory agent, an antiviral agent, an ion channel blocking agent, and an opioid.

54. The composition of claim 53, wherein the base composition comprises 0.1% to 30% (w/w) of one or more therapeutic agents.

55. The composition of claim 54, wherein the base composition comprises 1% to 10% (w/w) of one therapeutic agent.

56. The composition of claim 54, wherein the base composition comprises 10% to 25% (w/w) of two or more therapeutic agents.

57. The composition of claim 53, wherein the one or more therapeutic agents are one or more antihistamines.

58. The composition of claim 57, wherein the one or more antihistamines are from 1% to 25% (w/w) of one or more of doxepin, amitriptyline, triprolidine, acrivastine, or diphenhydramine or a pharmaceutically acceptable salt thereof.

59. The composition of claim 53, wherein the one or more therapeutic agents are the antihistamine and the antiinflammatory agent.

60. The composition of claim 59, wherein the antihistamine is 1% to 10% (w/w) of doxepin or a pharmaceutically acceptable salt thereof and the antiinflammatory agent is 1% to 10% (w/w) of ketoprofen.

61. The composition of claim 53, wherein the one or more therapeutic agents are the antihistamine and the antiviral agent.

62. The composition of claim 61, wherein the antihistamine is from 1% to 10% (w/w) doxepin or a pharmaceutically acceptable salt thereof and the antiviral agent is from 5% to 15% (w/w) acyclovir or valacyclovir.

63. The composition of claim 53, wherein the one or more therapeutic agents are the antihistamine and the ion channel blocking agent.

64. The composition of claim 61, wherein the antihistamine is from 1% to 10% (w/w) doxepin or a pharmaceutically acceptable salt thereof and the ion channel blocking agent is from 5% to 15% (w/w) lidocaine, benzocaine, bupivacaine, etidocaine, mepivacaine, or tetracaine.

65. The composition of claim 53, further comprising a skin penetration enhancer.

66. The composition of claim 65, wherein the skin penetration enhancer is selected from the group consisting of a polyacrylic acid polymer, a polysaccharide gum, isopropyl myristate, isopropyl palmitate, dimethyl sulfoxide, decyl methyl sulfoxide, dimethylalanine amide of a medium chain fatty acid, dodecyl 2-(N,N-dimethylamino) propionate, tetradecyl (N,N-dimethylamino) acetate, dodecyl (N,N-dimethylamino) acetate, decyl (N,N-dimethylamino) acetate, octyl (N,N-dimethylamino) acetate, and dodecyl (N,N-diethylamino) acetate, or salts thereof.

67. The composition of claim 48, further comprising a skin penetration enhancer.

68. The composition of claim 67, wherein the skin penetration enhancer is selected from the group consisting of a polyacrylic acid polymer, a polysaccharide gum, isopropyl myristate, isopropyl palmitate, dimethyl sulfoxide, decyl methyl sulfoxide, dimethylalanine amide of a medium chain fatty acid, dodecyl 2-(N,N-dimethylamino) propionate, tetradecyl (N,N-dimethylamino) acetate, dodecyl (N,N-dimethylamino) acetate, decyl (N,N-dimethylamino) acetate, octyl (N,N-dimethylamino) acetate, and dodecyl (N,N-diethylamino) acetate, or salts thereof.

69. The composition of claim 48, wherein the composition is formulated as a cream, a gel, a lotion, an ointment, or a liquid.

70. A kit comprising the composition of claim 48, instructions for administering the composition to a subject, and an applicator for applying the composition.

71. A kit comprising the composition of claim 53, instructions for administering the composition to a subject, and an applicator for applying the composition.
